(19) United States
(12) Patent Application Publication
(10) Pub. No.: US 2005/0187494 A1
(43) Pub. Date: Aug. 25, 2005

(54) Focusing ultrasonic source

(76) Inventors: Shenxu He, Beijing (CN); Jinsheng Yu, Beijing (CN); Jiang Lan, Beijing (CN); Xiaodong Wu, Beijing (CN)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/494,531

(22) PCT Filed: Mar. 15, 2002
(86) PCT No.: PCT/CN02/00169

(30) Foreign Application Priority Data
Nov. 5, 2001 (CN) 01134484.9

(51) Int. Cl. A61N 7/00

(52) U.S. Cl. 601/2

(57) ABSTRACT

The present invention provides a focus ultrasonic wave source including an ultrasonic wave emitting component for emiting the ultrasonic wave and a focusing component for focusing the the emited ultrasonic wave, wherein the emitted ultrasonic wave is transmitted to a focal point in a manner similar to a spherical wave after it is focused by said focusing component, and wherein an angle α included between the two lines connecting two end points of the outer peripheral diameter of said focusing component to the focal point respectively is from 50° to 120°. After tested, it is found that the ultrasonic wave source according to the present invention has a focusing performance substantilly superior to that of the existing ultrasonic wave source with a small aperture angle, and it can cause the focused ultrasonic wave to reach a sufficiently high sound intensity and temperature at the focal point so as to kill the body tissues while avoiding or reducing the damages to other tissue of the human body in the ultrasonic wave transmission path, which greatly reduces the pain felt by the patient in clinical applications and achieves a desired effect.

### FIELD OF THE INVENTION

The present invention relates to a means for use in a medical apparatus, especially to an ultrasonic wave source for use in a high intensity focus ultrasonic wave (HIFU) therapeutic apparatus.

### BACKGROUND OF THE INVENTION

At present, the existing extracorporeal HIFU therapeutic apparatus generally includes the following means: a HIFU source and a driving circuit thereof for generating the HIFU; a locating system for searching the object to be treated and moving it to the focal point of the ultrasonic transducer, which includes a medical imaging system (such as the B-Mode Ultrasonoscope), a carrying device for carrying the patient such as a bed surface, and a displacement system for causing the spatial movement of the carrying device with respect to the ultrasonic wave source; a high intensity ultrasonic wave (HIU) transmitter and a processing system for transmission medium. Since the ultrasonic wave adapted to the HIFU must be transmitted into the patient body through a special transmission medium (such as deaerated water), a containing means for receiving the transmission medium (such as a water tank or a water bag etc.) in front of the emitting surface of the HIFU source and means for filling, discharging and processing the transmission medium are necessary.

Among the above-mentioned means of the extracorporeal HIFU therapeutic apparatus, the HIFU source is the most important. Disregard of the focusing manner (such as refraction focusing of a lens, reflection focusing of a curved surface and self focusing of a concave spherical surface or the like), the ultrasonic wave emitted by the focus ultrasonic wave source is always transmitted to the focal point in a manner similar to a spherical wave after the wave is focused. Herein, the outer peripheral diameter of the emitting surface of the focus ultrasonic wave source is referred to as an “aperture diameter”, and the angle included between the two lines connecting the two end points of the outer peripheral diameter thereof to the focal point respectively is referred to as the “aperture angle”.

The contradiction between the safety and the effectiveness is the primal problem to be solved in the field of the medical apparatus. Much research and pratice on the HIFU therapeutic apparatus has proved that it is not difficult to kill the tissues by means of sufficiently high sound intensity and temperature obtained at the focal point by the HIFU. However, in this case, it is difficult to avoid or reduce the damages to other tissue in the human body in the ultrasonic wave transmission path. Whether the HIFU therapy is successful or not is determined by the above problem. Improving the focusing performance of the HIU source is the main way to solve the above problem while the aperture angle is the main parameter in relation to the focusing performance of the ultrasonic wave source.

The ultrasonic wave sources of most of the existing HIFU therapeutic apparatus have an aperture angle of 60°, and some ultrasonic wave sources have a even less aperture angle (less than 50°). According to the present pratice, the focusing performance of the ultrasonic wave sources with such aperture angles is not desired. Specifically, such wave sources will make the patient feel an evident pain and cause the damage in the transmission path, or they can not reach sufficiently high sound intensity and temperature at the focal point for killing the tissues.

The percentage ratio of the maximum sound pressure in the ultrasonic wave path within the range from 3 to 5 cm in front of the focal point to the sound pressure at the focal point is considered as the index for assessing the focusing performance to research the realtionship between the aperture angle and the ratio (the less the ratio is, the better the focusing performance is). It has been proved that the focusing performance is substantially improved with the increase of the aperture angle when the aperture angle is relatively little, and the improvement of the focusing performance with the increase of the aperture angle is gradually suppressed when the aperture angle increases to a certain degree. In addition, becase of the increase of the size and cost of the ultrasonic wave source and the difficulty in therapy due to the increase of the aperture angle, it is necessary to solve the above problem by setting the aperture angle of the focusing component of the ultrasonic wave source in a desired angle range.

### SUMMARY OF THE INVENTION

The object of the present invention is to provide a focus ultrasonic wave source with a desired focusing performance. The ultrasonic wave source can generate the ultrasonic wave focused to reach a sufficiently high sound intensity and temperature at the focal point so as to kill the tissues while avoiding or reducing the damages to other tissue of the human body in the ultrasonic wave transmission path.

In order to fulfill the above-mentioned object, the present invention provides a focus ultrasonic wave source, which includes an ultrasonic wave emitting component for emiting the ultrasonic wave and a focusing component for focusing the the emited ultrasonic wave, wherein the emitted ultrasonic wave is transmitted to the focal point in a manner similar to a spherical wave after it is focused by said focusing component, and wherein an angle included between the two lines connecting two end points of the outer peripheral diameter of said focusing component to the focal point respectively is from 50° to 120°.

Preferably, the angle included between the two lines connecting two end points of the outer peripheral diameter of said focusing component to the focal point respectively is from 50° to 60°, such as 56°, or from 60° to 120°, such as 110°.

After tested, it is found that the ultrasonic wave source according to the present invention has a focusing performance substantilly superior to that of the existing ultrasonic wave source with a small aperture angle, and it can cause the focused ultrasonic wave to reach a sufficiently high sound intensity and temperature at the focal point so as to kill the tissues while avoiding or reducing the damages to other tissue of the human body in the ultrasonic wave transmission path, which greatly reduces the pain felt by the patient.

### BRIEF DESCRIPTION OF DRAWINGS

The present invention will be described in detail by the preferred embodiments with reference to the following drawings, wherein,

FIG. 1 is a schematic view of one embodiment of the focus ultrasonic wave source according to the present invention, the focusing manner of which is spherical self focusing;

FIG. 2 is a schematic view of another embodiment of the focus ultrasonic wave source according to the present invention, the focusing manner of which is lens focusing.

### THE BEST MODE OF THE EMBODIMENTS

The drawings schematically show the structural principle views of a focus ultrasonic wave source according to the present invention, which includes an ultrasonic wave emitting component 2 for emiting the ultrasonic wave and a focusing component 1 for focusing the emitted ultrasonic wave, wherein the emitted ultrasonic wave is transmitted to a focal point F in a manner similar to a spherical wave after it is focused by said focusing component 1, and wherein an angle α (i.e. “aperture angle” mentioned above) included between the two lines connecting two end points of the outer peripheral diameter of said focusing component 1 to the focal point respectively is from 50° to 120°.

Preferably, the angle (aperture angle) a included between the two lines connecting two end points of the outer peripheral diameter of said focusing component to the focal point respectively is from 50° to 60°, such as 56°, or the angle (aperture angle) α is from 60° to 120°, such as 110°.

For example, the aperture angle α of the ultrasonic wave source is 56°, the focusing manner thereof is self focusing of multiple concave spherical surfaces and the focusing radius R is 300 mm. The ultrasonic wave source can be used in an HIFU therapeutic apparatus with a lower-mounted ultrasonic wave source.

Alternatively, for example, the aperture angle α of the ultrasonic wave source is 110°, the focusing manner thereof is concave spherical focusing and the focusing radius R is 50 mm. The ultrasonic wave source can be used to treat the shallow portion in the body by HIFU.

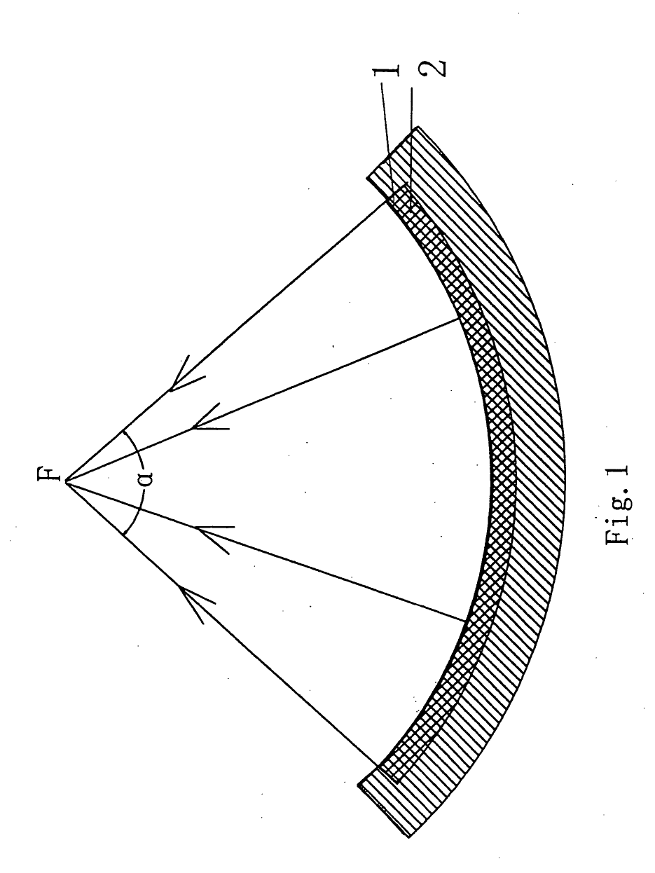

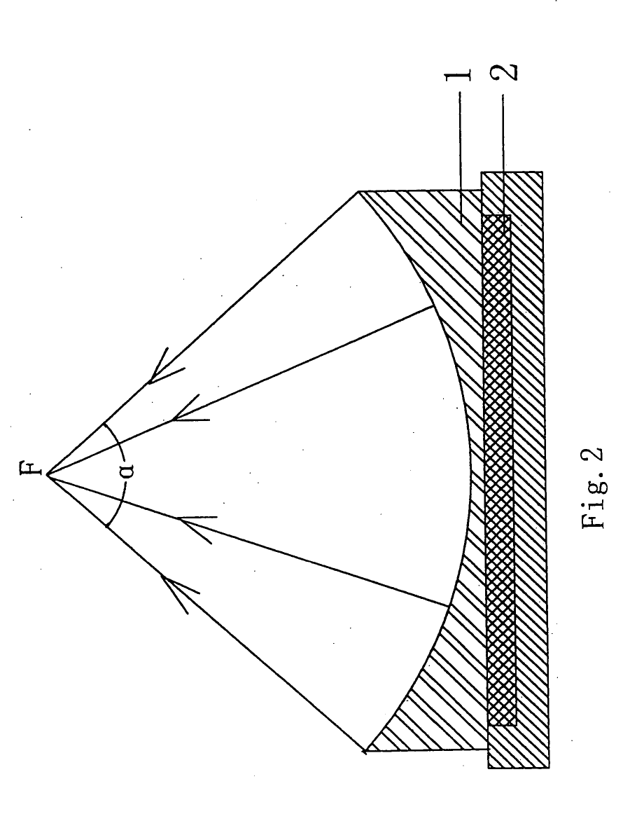

As shown in FIG. 2, the focusing component 1 may be a lens which focuses the waves in a manner of refraction. Alternatively, as shown in FIG. 1, the focusing component 1 may be a concave spherical surface which focuses the waves in a manner of self focusing.

As mentioned above, it is preferable that the aperture angle of the focusing component of the ultrasonic wave source is from 50° to 120°. That is, in the case of different applications, the aperture angle is from 50° to 60°, or from 60° to 120°. For teating the shallow portion in the human body, the preferable aperture angle is more than 60°.

After tested, the ultrasonic wave source according to the present invention has a focusing performance substantilly superior to that of the existing ultrasonic wave source with a small aperture angle, and it can cause the focus ultrasonic wave to reach a sufficiently high sound intensity and temperature at the focal point so as to kill the tissues while avoiding or reducing the damages to other tissue in the human body in the ultrasound transmission path, which greatly reduces the pain felt by the patient in clinical applications and achieves a desired effect.

1. A focus ultrasonic wave source including an ultrasonic wave emitting component (2) for emiting the ultrasonic wave and a focusing component (1) for focusing the the emited ultrasonic wave, the emitted ultrasonic wave being transmitted to a focal point F in a manner similar to a spherical wave after it is focused by said focusing component (1), wherein an angle (α) included between the two lines connecting two end points of the outer peripheral diameter of said focusing component (1) to the focal point respectively is from 50° to 120°.

2. A focus ultrasonic wave source according to claim 1, wherein the angle (α) included between the two lines connecting two end points of the outer peripheral diameter of said focusing component (1) to the focal point respectively is from 50° to 60°.

3. A focus ultrasonic wave source according to claim 2, wherein the angle (α) included between the two lines connecting two end points of the outer peripheral diameter of said focusing component (1) to the focal point respectively is 56°.

4. A focus ultrasonic wave source according to claim 1, wherein the angle (α) included between the two lines connecting two end points of the outer peripheral diameter of said focusing component (1) to the focal point respectively is from 60° to 120°.

5. A focus ultrasonic wave source according to claims 4, wherein the angle (α) included between the two lines connecting two end points of the outer peripheral diameter of said focusing component (1) to the focal point respectively is 110°.

6. A focus ultrasonic wave source according to any one of claims 1-4, wherein said focusing component (1) is a lens which focuses the waves in a manner of refraction.

7. A focus ultrasonic wave source according to any one of claims 1-4, wherein said focusing component (1) is a curved surface which focuses the waves in a manner of reflection.

8. A focus ultrasonic wave source according to any one of claims 1-4, wherein said focusing component (1) is a concave spherical surface which focuses the waves in a manner of self focusing.
